(19) United States
(12) Patent Application Publication
(10) Pub. No.: US 2023/0390298 A1
(43) Pub. Date: Dec. 7, 2023

(54) METHOD AND COMPOSITIONS FOR USE IN PROVIDING MALE CONTRACEPTION

(71) Applicant: Eppin Pharma, Inc., Chapel Hill, NC (US)

(72) Inventors: Michael G. O'Rand, Chapel Hill, NC (US); Katherine G. Hamil, Chapel Hill, NC (US)

(73) Assignee: Eppin Pharma, Inc., Chapel Hill, NC (US)

(21) Appl. No.: 17/833,131

(22) Filed: Jun. 6, 2022

(51) Int. Cl. A61K 31/53; A61K 31/44; A61K 31/505

(52) U.S. Cl. CPC A61K 31/53; A61K 31/44; A61K 31/505

(57) ABSTRACT

A method and compositions suitable for use in providing male contraception by inhibiting the forward motility and function of sperm in humans and other mammals.

### STATEMENT REGARDING FEDERALLY SPONSORED RESEARCH OR DEVELOPMENT

This invention was funded by the National Institutes of Health, NICHD Chemical Screening and Optimization facility, government contract number HHSN2752018000071.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

### Field of the Invention

The present invention relates to a method and compositions for providing male contraception. In particular, it relates to a method and compositions which can inhibit the forward motility and impair the function of sperm in humans and other mammals.

### Description of Related Art

Over the last 50 years, contraception has had a major impact on human society and influenced the worldwide distribution of family sizes and the variability of fertility rates (Bongaarts and Watkins, 1996; Bongaarts, 1997). This impact can be largely attributed to female contraceptive methods, their availability, and economic and social costs.

Male contraception, on the other hand, has had much less of a global impact, being largely limited to condoms and vasectomy (Nass and Strauss, 2004). Female hormonal contraceptives work through the mechanism of anovulation and the goal of male hormonal contraceptive research is analogous, namely the suppression of spermatogenesis to produce azoospermia. However, achievement of this goal in a reliable way for a diverse population of men is still many years away (Grimes et al., 2005; Potts, 1996).

Even further away is the dream of a non-hormonal male contraceptive in which it may be envisioned that spermatozoa do not develop, or do not swim, or do not fertilize or some combination of these spermatozoan catastrophes. Numerous contraceptive targets abound and several of these targets are worthy of further exploratory work, including blocking transmembrane ion currents (Kirichok et al., 2006; Brenton et al., 1996), disrupting Sertoli-germ cell adhesions (Cheng et al., 2002, 2005) and disruption of spermiogenesis by imino sugars (Walden et al., 2006).

Immunocontraception, which showed great promise for many years, has lost its appeal. Nevertheless, immunocontraception can be used as a strategy to discern the function of target molecules in the male. As an example, EPPIN is an epididymal protease inhibitor that coats the surface of human spermatozoa. EPPIN modulates PSA (prostate specific antigen, a serine protease) activity and the hydrolysis of semenogelin (SEMG1). Although EPPIN modulates the hydrolysis of semenogelin by PSA, antibodies to EPPIN do not inhibit PSA activity.

Ejaculate spermatozoa of monkeys and humans are coated with EPPIN. On the surface of spermatozoa, EPPIN binds the protein semenogelin, which is secreted by the seminal vesicles during ejaculation. The EPPIN-semenogelin complex is dissociated during liquefaction of semen during the first 30 minutes after ejaculation due to the cleavage of SEMG1 by PSA to small fragments that no longer interact with EPPIN. Failure to remove semenogelin results in infertile spermatozoa. Studies of the interaction of EPPIN (and semenogelin) and their effect on human spermatozoa are described, for example, in Wang, Z., Widgren, E. E., Sivashanmugam, P., O'Rand, M. G., and Richardson, R. T. 2005. Association of EPPIN with semenogelin on human spermatozoa, Biology of Reproduction 72 (4): 1064-1070 (Dec. 8, 2004).

One strategy for developing new contraceptives is to immunize mammals with specific sperm surface antigens and determine the effects of the immune response on the ejaculated spermatozoa of immunized males. Past work on EPPIN, (SPINLW1; serine protease inhibitor-like, with Kunitz and WAP domains-1) provides an example of the utility of the immunocontraceptive approach (O'Rand et al., 2004; Wang et al., 2005; O'Rand et al., 2006). A fertility study (O'Rand et al., 2004) demonstrated that effective and reversible male immunocontraception in mammals is an obtainable goal. A high serum titer (>1:1000) sustained over several months achieved an effective level of contraception. Treatment of human spermatozoa with antibodies to EPPIN derived from mammals showed a decrease in motility of the treated spermatozoa, (results are described, for example, in O'Rand, M. G., Widgren, E. E., Beyler, S. and Richardson, R. T. 2009). Inhibition of human sperm motility by contraceptive anti-EPPIN antibodies from infertile male monkeys: effect on cyclic adenosine monophosphate, Biology of Reproduction 80: 279-285 (Oct. 22, 2008).

The development of a non-hormonal male contraceptive can enhance family planning throughout the world and give men and women additional contraceptive choices. Currently, men are limited in their options for contraception to condoms and vasectomies. In recent surveys, the satisfaction rate for women on contraception is less than 60% for every method except tubal ligation, and men want access to better contraceptives. Therefore, a non-hormonal male contraceptive will fill an unmet need in contraception.

### BRIEF SUMMARY OF THE INVENTION

The present invention relates to a method of providing contraception using the compositions that are provided and compositions suitable for use in providing male contraception by inhibiting the forward motility and impairing the function of sperm in humans and other primates.

Accordingly, in one embodiment there is a method of providing male contraception, comprising administering an effective amount of a small molecule that mimics the binding of anti-EPPIN antibodies to EPPIN, to a patient in need of male contraception selected from the group consisting of:
a) methyl 3-({4-[(4-acetamido-3-hydroxyphenyl)amino]-6-{[(methoxycarbonyl)amino]amino}-1,3,5-triazin-2-yl}sulfanyl)propanoate;
b) 3-((4-((4-acetamido-3-hydroxyphenyl)amino)-6-(2-(methoxycarbonyl)hydrazineyl)-1,3,5-triazin-2-yl)thio)propanoic acid;
c) methyl 2-[(4{[3-(acetyloxy)-4 acetamidophenyl]amino}-6-{[(methoxycarbonyl)amino]amino}pyrimidin-2-yl)sulfa acetate;
d) methyl 2-(4-((4-acetamido-3-hydroxyphenyl)amino)-6-((3-oxobutyl)thio)-1,3,5-triazin-2-yl) hydrazine-1-carboxylate;
e) 4-((4-((4-acetamido-3-hydroxyphenyl)amino)-6-(2-(methoxycarbonyl)hydrazineyl)-1,3,5-triazin-2-yl)thio)butanoic acid;
f) methyl 2-({4[(4 acetamido-3-hydroxyphenyl)amino]-6-{[(methoxycarbonyl)amino]amino}pyrimidin-2-yl}sulfan acetate;
g) methyl 4-({4-[(4-acetamido-3-hydroxyphenyl)amino]-6-{[(methoxycarbonyl)amino]amino}-1,3,5-triazin-2-yl}sulfanyl)butanoate;
h) methyl 3-({2-[(4-acetamido-3-hydroxyphenyl)methyl]-6-{[(methoxycarbonyl)amino]amino}pyrimidin-4-yl}sulfanyl)propanoate;
i) methyl 3-[(6-{[3-hydroxy-4-(2-oxopropyl)phenyl]methyl}-4-{[(methoxycarbonyl)amino]amino}pyridin-2-yl)sulfanyl]propanoate;
j) methyl 3-{[2-({3-hydroxy-4-[(1E)-(methoxyimino)methyl]phenyl}methyl)-6-{[(methoxycarbonyl)amino]amino}pyrimidin-4-yl]sulfanyl}propanoate;
k) methyl 3-{[6-({3-hydroxy-4-[(1E)-(methoxyimino)methyl]phenyl}methyl)-4-{[(methoxycarbonyl)amino]amino}pyridin-2-yl]sulfanyl}propanoate; and
l) 3-{[6-({3-hydroxy-4-[(1E)-(methoxyimino)methyl]phenyl}methyl)-4-{[(methoxycarbonyl)amino]amino}pyridin-2-yl]sulfanyl}propanoic acid.

In another embodiment, there is a composition providing male contraception comprising administering an effective amount of one or more compositions, the compositions consisting of:
a) methyl 3-({4-[(4-acetamido-3-hydroxyphenyl)amino]-6-{[(methoxycarbonyl)amino]amino}-1,3,5-triazin-2-yl}sulfanyl)propanoate;
b) 3-((4-((4-acetamido-3-hydroxyphenyl)amino)-6-(2-(methoxycarbonyl)hydrazineyl)-1,3,5-triazin-2-yl)thio)propanoic acid;
c) methyl 2-[(4{[3-(acetyloxy)-4 acetamidophenyl]amino}-6-{[(methoxycarbonyl)amino]amino}pyrimidin-2-yl)sulfa acetate;
d) methyl 2-(4-((4-acetamido-3-hydroxyphenyl)amino)-6-((3-oxobutyl)thio)-1,3,5-triazin-2-yl) hydrazine-1-carboxylate;
e) 4-((4-((4-acetamido-3-hydroxyphenyl)amino)-6-(2-(methoxycarbonyl)hydrazineyl)-1,3,5-triazin-2-yl)thio)butanoic acid;
f) methyl 2-({4[(4 acetamido-3-hydroxyphenyl)amino]-6-{[(methoxycarbonyl)amino]amino}pyrimidin-2-yl}sulfan acetate;
g) methyl 4-({4-[(4-acetamido-3-hydroxyphenyl)amino]-6-{[(methoxycarbonyl)amino]amino}-1,3,5-triazin-2-yl}sulfanyl)butanoate;
h) methyl 3-({2-[(4-acetamido-3-hydroxyphenyl)methyl]-6-{[(methoxycarbonyl)amino]amino}pyrimidin-4-yl}sulfanyl)propanoate;
i) methyl 3-[(6-{[3-hydroxy-4-(2-oxopropyl)phenyl]methyl}-4-{[(methoxycarbonyl)amino]amino}pyridin-2-Asulfanyl]propanoate;
j) methyl 3-{[2-({3-hydroxy-4-[(1E)-(methoxyimino)methyl]phenyl}methyl)-6-{[(methoxycarbonyl)amino]amino}pyrimidin-4-yl]sulfanyl}propanoate;
k) methyl 3-{[6-({3-hydroxy-4-[(1E)-(methoxyimino)methyl]phenyl}methyl)-4-{[(methoxycarbonyl)amino]amino}pyridin-2-yl]sulfanyl}propanoate; and
l) 3-{[6-({3-hydroxy-4-[(1E)-(methoxyimino)methyl]phenyl}methyl)-4-{[(methoxycarbonyl)amino]amino}pyridin-2-yl]sulfanyl}propanoic acid.

### BRIEF DESCRIPTION OF THE DRAWINGS

FIG. 1 is a standard epitope ALPHA dose response assay for 2-((4-((4-acetamido-3-hydroxyphenyl)amino)-6-(2-(methoxycarbonyl)hydrazinyl)-1,3,5-triazi n-2-yl)thio)acetic acid, 3-((4-((4-acetamido-3-hydroxyphenyl)amino)-6-(2-(methoxycarbonyl)hydrazineyl)-1,3,5-triazin-2-yl)thio)propanoic acid, methyl 2-(4-((4-acetamido-3-hydroxyphenyl)amino)-6-((3-oxobutyl)thio)-1,3,5-triazin-2-yl) hydrazine-1-carboxylate.

FIG. 2 is a standard epitope ALPHA dose response assay for 2-((4-((4-acetamido-3-hydroxyphenyl)amino)-6-(2-(methoxycarbonyl)hydrazinyl)-1,3,5-triazi n-2-yl)thio)acetic acid, 3-((4-((4-acetamido-3-hydroxyphenyl)amino)-6-(2-(methoxycarbonyl)hydrazineyl)-1,3,5-triazin-2-yl)thio)propanoic acid, and methyl 2-(4-(4-acetamido-3-hydroxyphenyl)amino)-6-((3-oxobutyl)thio)-1,3,5-triazin-2-yl) hydrazine-1-carboxylate.

FIG. 3 is a standard epitope ALPHA dose response assay for 4-((4-((4-acetamido-3-hydroxyphenyl)amino)-6-(2(methoxycarbonyl)hydrazineyl)-1,3,5-triazin-2-yl)thio)butanoic acid.

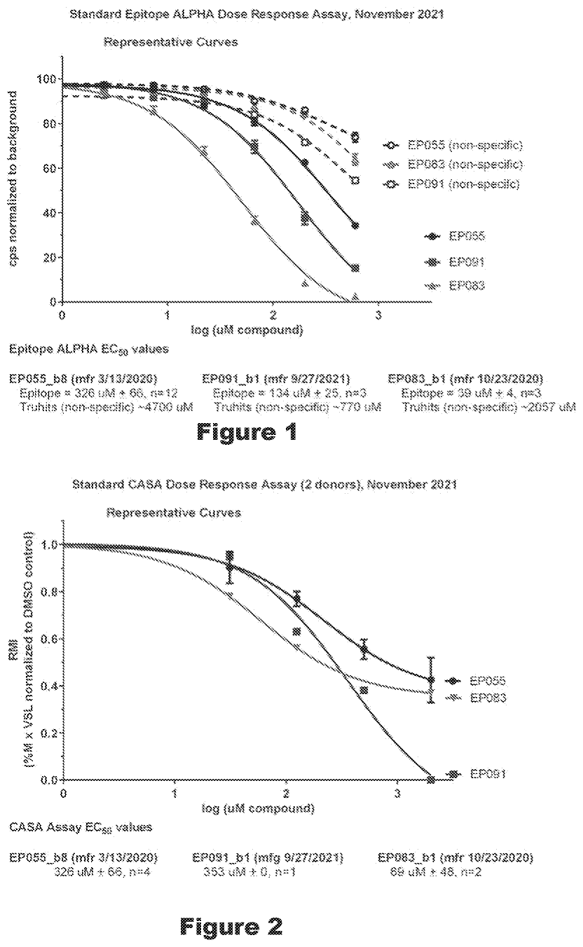

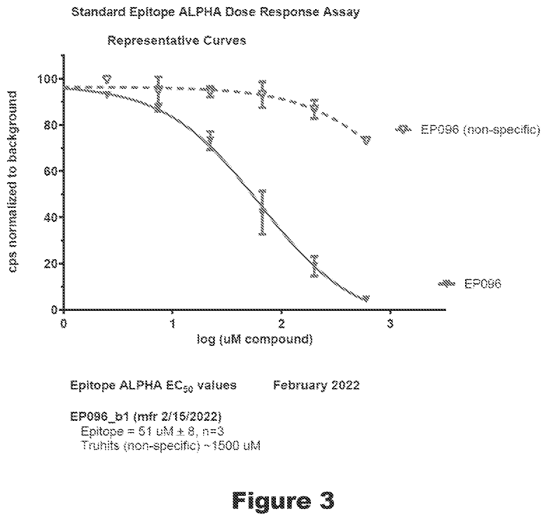

The present invention will be better understood with reference to the following detailed description.

### DETAILED DESCRIPTION OF THE INVENTION

EPPIN (SPINLW1; epididymal protease inhibitor) coats the surface of human testicular, epididymal, and ejaculate spermatozoa in an EPPIN protein complex (EPC) containing lactotransferrin and clusterin. EPPIN is a target for the assays described herein.

During ejaculation, semenogelin (SEMG1) binds to EPPIN in the complex, inhibiting the progressive motility and impairing the function of ejaculate spermatozoa. The EPPIN-semenogelin complex is on the surface of sperm. Subsequently, SEMG1 is hydrolyzed by the serine protease PSA, and EPPIN modulates PSA hydrolysis of SEMG1 on the sperm surface, resulting in forwardly motile spermatozoa.

Previous studies on the antisera from the infertile monkeys revealed two linear B-cell epitopes of anti-EPPIN, one in the N-terminal and one in the C-terminal. Antibodies to this epitope inhibit sperm motility and semenogelin binding, as well as impair the motility of sperm.

Immunocontraception (i.e., the use of antibodies to bring about contraception) is not considered a viable option for a marketable product for efficacy, safety, and economic reasons. Accordingly, in one embodiment, the invention relates to small organic compounds that mimic an anti-EPPIN antibody (i.e., compounds that bind EPPIN in the same position or substantially the same position as anti-EPPIN antibodies, and thus act as small molecules, (mimics for these antibodies), and thus inhibit the forward motility of sperm and impair function, and methods for their use in providing male contraception. These compounds can be administered orally, for example, and taken on-demand a relatively short time before ejaculation.

Based on a publication in Science, in 2004, demonstrating that blocking EPPIN-SEMG1 interaction results in the complete and reversible contraception of male monkeys immunized to a high titer with EPPIN, small organic lead compounds were developed as a sperm-based contraceptive. Accordingly, in one embodiment, the compounds inhibit semenogelin binding to EPPIN. Small organic compounds that inhibit sperm motility and impair such function by fitting into the EPPIN-SEMG1 binding site on the surface of spermatozoa can be administered orally, and taken on-demand a relatively short time before ejaculation.

In this embodiment, useful compounds include those that a) bind to the binding site on EPPIN for semenogelin (also referred to herein as SEMG1, or b) which bind to an allosteric position in a manner which inhibits semenogelin from binding, and which also mimic the effect of the semenogelin, namely to stop sperm from swimming. Ideally, those compounds which interfere with EPPIN semenogelin binding will bind with higher affinity to the active binding pocket than semenogelin. This will enable one to administer lower effective concentrations of the compounds than compounds that bind with lower binding affinity. Those compounds which bind in an allosteric manner are also ideally, high affinity compounds, so that lower effective concentrations of these compounds can be administered as well. In one embodiment, the compounds bind to the same position as anti-EPPIN antibodies, and thus act as small molecule mimics for these antibodies. In another embodiment, the compounds inhibit sperm motility and impair such function by inhibiting EPPIN-semenogelin binding.

Two assays for high throughput screening (HTS) of compounds have been established and validated. In one embodiment, these assays are based on an adaptation of the AlphaScreen™ assay developed by PerkinElmer (Waltham, MA). In the AlphaScreen™ (amplified luminescent proximity homogeneous assay) assay donor and acceptor beads (˜200 nm) are employed to hold interacting protein molecules. When the interacting protein molecules bind (for example EPPIN {on the donor bead} and anti-EPPIN {on the acceptor bead}), single state oxygen molecules diffuse from the donor bead to the acceptor bead (˜4 μsec) and fluorophores subsequently emit light at 520-620 nm. In the primary compound screen, histidine-tagged recombinant human EPPIN is attached to NTA-donor beads and anti-EPPIN (S21C; against the EPPIN C-terminal domain) is attached to protein A-acceptor beads. This epitope specific assay gives strong and stable binding between EPPIN and the antibody.

Once the activity of the compounds is determined, it can also be important to determine other pharmacological properties, including adsorption, distribution, metabolism, excretion, and toxicology. Any of a number of known screening assays can be used for this purpose. For example, a cytotoxicity assay called CellTiter-Glo®, a Luminescent Cell Viability Assay from Promega Corporation that is an automated high-throughput screen for cell proliferation and cytotoxicity can be used to determine the toxicity of the compounds.

In another embodiment, a computer-assisted sperm analysis (CASA) can be used as a live cell compound screen for determining the effect of compounds on human sperm motility. Additionally, to facilitate the EC₅₀ evaluation of compounds using different ejaculates, reducing inter-assay variation due to differences in sperm quality in different semen samples, an index of relative motility inhibition (RMI) was developed. This is calculated as: RMI=[% motility*VSL]; percentage of motile sperm (% motility) or the percentage of progressively motile sperm (% progressive motility, RPMI) multiplied by the straight-line velocity (VSL); the average velocity measured in a straight line from the beginning to the end of a track in μm/sec. Normalized RMI can be calculated by dividing the RMI of each experimental condition by its respective DMSO control.

Twelve compounds were identified as new modifications of EP055.

In vivo, the contraceptive drug must bind to EPPIN in the epididymis and either binds to the site at which the anti-EPPIN antibodies would bind, compete with semenogelin (SEMG1) for binding to EPPIN on the surface of sperm during ejaculation, or enhance the binding of semenogelin to EPPIN. The calculated EC₅₀ value for SEMG1 inhibiting sperm motility and impair proper function is 8.8 μM. Active compounds ideally have an EC₅₀ value within an order of magnitude of this EC₅₀ value, though compounds with higher and lower EC₅₀ values can be useful, so long as they can be safely administered at effective dosages.

The following 12 compounds demonstrate inhibitory activity in the both above-described assays: EP082, EP083, EP089, EP091, EP096, EP097, EP098, EP100, EP101, EP102, EP103, EP104. Effects on sperm are detectable within minutes. It is expected that the effect on motility in vivo will be equivalent or better to that of SEMG1 as sperm will be exposed to compound in the epididymis before exposure to SEMG1 during ejaculation

Lipinski's rule of five is a rule of thumb to evaluate whether a compound has properties that would make it a likely orally active drug in humans. The rule describes molecular properties important for a drug's pharmacokinetics in the human body, including their adsorption, distribution, metabolism, and excretion (“ADME”). According to this rule, a compound should have no more than five hydrogen bond donors (the total number of nitrogen—hydrogen and oxygen—hydrogen bonds), not more than ten hydrogen bond acceptors (all nitrogen or oxygen atoms), a molecular mass less than 500 Daltons, and an octanol-water partition coefficient log P not greater than five. Accordingly, it is believed that the compounds described herein will be orally bioavailable.

The compounds can be included in compositions, which ideally are oral or transdermal compositions, which release an appropriate amount of the compounds to produce this effect. For example, the compounds can be used in once-daily tablets or pills, or transdermal patches or subcutaneous implants for periods of time longer than a day, much in the same manner as female contraceptives.

In another embodiment, the compounds can be used in addition to, or in place of, spermicides in spermicidal compositions, such as those used in conjunction with condoms, diaphragms, and spermicidal jellies. That is, since the compounds can function on contact with spermatozoa to inhibit forward motility and impair function, it is not necessary that they be ingested to have the effect.

Thus, the invention described herein provides an advantage over the prior art, in that the user has a choice of male contraception between in vivo activity of the compounds to inhibit the forward motility of spermatozoa before ejaculation, or use of the compound after ejaculation to inhibit forward motility and proper function of the spermatozoa.

In addition to compounds that mimic the anti-EPPIN antibodies, it has been found that certain compounds bind EPPIN in the same site as semenogelin (as opposed to an allosteric position), and bind more tightly than semenogelin, thus inhibiting formation of an EPPIN-semenogelin complex. Other compounds bind EPPIN in an allosteric position, and inhibit EPPIN-semenogelin complex formation in that manner. Still other compounds enhance EPPIN-semenogelin complex formation. Any of these embodiments will work to inhibit spermatozoa forward motility and function.

Compounds that mimic the binding of an anti-EPPIN antibody to EPPIN, or which inhibit semenogelin from binding to EPPIN, and thus inhibit spermatozoa forward motility and function, can be used to temporarily and reversibly cause male infertility.

While this invention is susceptible to embodiment in many different forms, there is shown in the drawings, and will herein be described in detail, specific embodiments with the understanding that the present disclosure of such embodiments is to be considered as an example of the principles and not intended to limit the invention to the specific embodiments shown and described. In the description below, like reference numerals are used to describe the same, similar, or corresponding parts in the several views of the drawings. This detailed description defines the meaning of the terms used herein and specifically describes embodiments in order for those skilled in the art to practice the invention.

### Definitions

The terms “about” and “essentially” mean±10 percent.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “comprising” is not intended to limit inventions to only claiming the present invention with such comprising language. Any invention using the term comprising could be separated into one or more claims using “consisting” or “consisting of” claim language and is so intended.

Reference throughout this document to “one embodiment”, “certain embodiments”, “an embodiment”, or similar terms means that a particular feature, structure, or characteristic described in connection with the embodiment is included in at least one embodiment of the present invention. Thus, the appearances of such phrases in various places throughout this specification are not necessarily all referring to the same embodiment. Furthermore, the particular features, structures, or characteristics may be combined in any suitable manner in one or more embodiments without limitation.

The term “or”, as used herein, is to be interpreted as an inclusive or meaning any one or any combination. Therefore, “A, B, or C” means any of the following: “A; B; C; A and B; A and C; B and C; A, B, and C”. An exception to this definition will occur only when a combination of elements, functions, steps, or acts are in some way inherently mutually exclusive.

The drawings featured in the figures are for the purpose of illustrating certain convenient embodiments of the present invention and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein, and use of the term “means” is not intended to be limiting.

EPPIN (SPINLW1; serine protease inhibitor-like, with Kunitz and WAP domains 1) is a member of the whey acidic protein (WAP)-type four-disulfide core (WFDC) gene family. The WFDC genes are on human chromosome 20q12-q13 in two clusters, one centromeric and one telomeric (Clauss et al., 2002). EPPIN is WFDC 7, also referred to as SPINLW1 (Gene ID: 57119), in the telomeric cluster and is the archetype of WFDC genes characterized by encoding both Kunitz-type and WAP-type four disulfide core protease inhibitor consensus sequences.

EPPIN is a testis/epididymal specific protein (Richardson et al., Gene 270, 93 (2001) and Sivashanmugam, et al., Gene 312, 125 (2003). The human EPPIN gene on chromosome 20 encodes two isoforms, one with and one without a secretory signal sequence, each containing both a Kunitz-type and a WAP-type (four-disulfide core) protease inhibitor consensus sequence. EPPIN represents the first member of a family of protease inhibitors on human chromosome 20 characterized by dual inhibitor consensus sequences (ibid). There are three splice variants of EPPIN that are expressed differently; EPPIN-1 is expressed in the testis and epididymis, EPPIN-2 is expressed in the epididymis and EPPIN-3 in the testis.

The preparation of recombinant human EPPIN (rhEPPIN) has been described in detail (Silva et al., Biology of Reproduction, 56:1-8 (2012) and the rhEPPIN used in the examples described herein lacks part of the N-terminal secretory signal sequence as described in Silva et al., ibid. Briefly, rhEPPIN was prepared in E. coli strain OragamiB-DE3-pLysS and the protein purified from the bacterial lysate on a Ni²⁺-NTA column (Qiagen, Valencia, Calif.; 21). The recombinant EPPIN used in the assays described herein has been tagged with a HISS tag.

Semenogelin I (SEMGI) and semenogelin II (SEMGII) are the dominating protein components of the coagulum formed by freshly ejaculated human semen. These proteins are primarily found in the seminal vesicles, although SEMGII is produced in small amounts in the epididymis. These proteins have not been detected in other tissues (Lundwall et al., Mol. Hum. Reprod.8 (9):805-10 (September 2002)).

As used herein, the terms “active ingredient” or “active agent” mean compounds which inhibit EPPIN-semenogelin binding and inhibit spermatozoa forward motility and function, as well as any prodrugs thereof and pharmaceutically acceptable salts, hydrates, and solvates of the compound and the prodrugs.

As used herein, the term “other ingredients” means any excipients, diluents, binders, lubricants, carriers, surfactants, and mixtures thereof that are formulated with the active compounds described herein, or any prodrugs thereof, and pharmaceutically acceptable salts, hydrates, and solvates thereof.

As used herein, the term “appropriate period of time” or “suitable period of time” means the period of time necessary to achieve a desired effect or result. For example, a mixture can be blended until a potency distribution is reached that is within an acceptable range for a given application or use of the blended mixture.

As used herein, the terms “carriers” or “vehicles” refer to carrier materials suitable for drug administration, specifically including oral and transdermal administration, and include any such materials known in the art (e.g., any liquid, gel, solvent, liquid diluent, solubilizer, or the like), which is non-toxic and which does not interact with other components of the composition in a deleterious manner. Examples of suitable vehicles for use in transdermal formulations include water, alcohols such as isopropyl alcohol and isobutyl alcohol, polyalcohols such as glycerol, and glycols such as propylene glycol, and esters of such polyols, (e.g., mono-, di-, or tri-glycerides).

As used herein, the term “controlled” means reduced or minimized peak and valley exposure cycles in blood, plasma, or other biological fluids normally present in some routes of administration of a pharmacologically active agent.

As used herein, the terms an “effective” or an “adequate” permeation enhancer for transdermal formulations is a permeation enhancer that will provide the desired increase in skin permeability and correspondingly, the desired depth of penetration, rate of administration, and amount of drug delivered.

As used herein, the terms “penetration enhancement” or “permeation enhancement” in connection with transdermal administration relates to an increase in the permeability of skin to a pharmacologically active agent, i.e., so as to increase the rate at which the drug permeates through the skin (i.e., flux) and enters the bloodstream. The enhanced permeation effected by using these enhancers can be observed by measuring the rate of diffusion (or flux) of drug through animal or human skin or a suitable polymeric membrane using a diffusion cell apparatus as described in the examples herein.

As used herein, the term “administer” or “administering” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, injectable and biodegradable in-situ forming implant (ISFI) or LARC (long acting reversible contraceptive), and topical (including buccal and sublingual) administration, injectable and biodegradable in-situ forming implant (ISFI) or LARC (long acting reversible contraceptive).

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro-spinal, and intrasternal injection and infusion. In preferred embodiments, the compositions are administered by intravenous infusion or injection.
Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compounds are formulated into ointments, salves, gels, or creams as generally known in the art.

As used herein, the term “sustained” means extended maintenance of steady state plasma levels of an active compound.

As used herein, the terms “unit dose”, “unit dosage”, or “unit dosage form” means a physically discrete unit that contains a predetermined quantity of active ingredient calculated to produce a desired therapeutic effect. The dosage form can be in any suitable form for administration, such as oral or transdermal administration, which forms are well known to those of skill in the art.

As used herein, the term “independently” indicates that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

As used herein, the term “alkyl”, unless otherwise specified, refers to saturated straight, branched, or cyclic, primary, secondary, or tertiary hydrocarbons, including both substituted and unsubstituted alkyl groups. The alkyl group can be optionally substituted with any moiety that does not otherwise interfere with the reaction or that provides an improvement in the process, including but not limited to halo, haloalkyl, hydroxyl, carboxyl, acyl, aryl, acyloxy, amino, amido, carboxyl derivatives, alkylamino, dialkylamino, arylamino, alkoxy, aryloxy, nitro, cyano, sulfonic acid, thiol, imine, sulfonyl, sulfanyl, sulfinyl, sulfamonyl, ester, carboxylic acid, amide, phosphonyl, phosphinyl, phosphoryl, phosphine, thioester, thioether, acid halide, anhydride, oxime, hydrozine, carbamate, phosphonic acid, phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference. Specifically included are CH₂F and CHF₂.

As used herein, whenever the term C (alkyl range) is used, the term independently includes each member of that class as if specifically, and separately set out. The term “alkyl” includes C₁₋₆ alkyl moieties. It is understood to those of ordinary skill in the art that the relevant alkyl radical is named by replacing the suffix “-ane” with the suffix “-yl”.

As used herein, the term “alkenyl” refers to an unsaturated, hydrocarbon radical, linear or branched, in so much as it contains one or more double bonds. The alkenyl group disclosed herein can be optionally substituted with any moiety that does not adversely affect the reaction process, including but not limited to those described for substituents on alkyl moieties. Non-limiting examples of alkenyl groups include ethylene, methylethylene, isopropylidene, 1,2-ethane-diyl, 1,1-ethane-diyl, 1,3-propane-diyl, 1,2 propane-diyl, 1,3-butane-diyl, and 1,4-butane-diyl.

As used herein, the term “alkynyl” refers to an unsaturated, acyclic hydrocarbon radical, linear or branched, in so much as it contains one or more triple bonds. The alkynyl group can be optionally substituted with any moiety that does not adversely affect the reaction process, including but not limited to those described above for alkyl moieties. Non-limiting examples of suitable alkynyl groups include ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, and hexyn-3-yl, 3,3-dimethylbutyn-1-yl radicals.

As used herein, the terms “alkylamino” or “arylamino” refers to an amino group that has one or two alkyl or aryl substituents, respectively.

As used herein, the term “protected” unless otherwise defined, refers to a group that is added to an oxygen, nitrogen, or phosphorus atom to prevent its further reaction or for other purposes. A wide variety of oxygen and nitrogen protecting groups are known to those skilled in the art of organic synthesis, and are described, for example, in Greene et al., Protective Groups in Organic Synthesis, supra.

As used herein, the term “aryl”, alone or in combination, means a carbocyclic aromatic system containing one, two or three rings wherein such rings can be attached together in a pendent manner or can be fused. Non-limiting examples of aryl include phenyl, biphenyl, or naphthyl, or other aromatic groups that remain after the removal of a hydrogen atom from an aromatic ring. The term aryl includes both substituted and unsubstituted moieties.

The aryl group can be optionally substituted with any moiety that does not adversely affect the compound synthesis, including but not limited to those described above for alkyl moieties.

In one embodiment, aryl groups also include C₅₋₁₀ heteroaryl rings, such as thiophene, pyrollidine, pyridine, and pyrimidine, which can be substituted in the same manner as the carbocyclic aryl rings.

As used herein, the terms “alkaryl” or “alkylaryl” refer to an alkyl group with an aryl substituent. More specifically, alkyl-aryl is a C₁₋₆ alkyl group bound to a C₅₋₁₀ aryl or heteroaryl ring, such as a benzyl group, and aryl-alkyl is a C₅₋₁₀ aryl or heteroaryl ring bound to a C₁₋₆ alkyl group.

As used herein, the term “halo,” as used herein, includes chloro, bromo, iodo and fluoro.

As used herein, the term “acyl” refers to a carboxylic acid ester in which the non-carbonyl moiety of the ester group is selected from straight, branched, or cyclic alkyl or lower alkyl, alkoxyalkyl including but not limited to methoxymethyl, aralkyl including but not limited to benzyl, aryloxyalkyl such as phenoxymethyl, aryl including but not limited to phenyl optionally substituted with halogen (F, Cl, Br, I), alkyl (including but not limited to C1, C2, C3, and C4) or alkoxy (including but not limited to C1, C2, C3, and C4), sulfonate esters such as alkyl or aralkyl sulphonyl including but not limited to methanesulfonyl, the mono, di or triphosphate ester, trityl or monomethoxytrityl, substituted benzyl, trialkylsilyl (e.g., dimethyl-t-butylsilyl) or diphenylmethylsilyl. Aryl groups in the esters optimally comprise a phenyl group.

As used herein, the term “lower acyl” refers to an acyl group in which the non-carbonyl moiety is lower alkyl.

As used herein, the terms “alkoxy” and “alkoxyalkyl” embrace linear or branched oxy-containing radicals having alkyl moieties, such as methoxy radical. The term “alkoxyalkyl” also embraces alkyl radicals having one or more alkoxy radicals attached to the alkyl radical, that is, to form monoalkoxyalkyl and dialkoxyalkyl radicals. The “alkoxy” radicals can be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

As used herein, the term “alkylamino” denotes “monoalkylamino” and “dialkylamino” containing one or two alkyl radicals, respectively, attached to an amino radical. The terms arylamino denotes “monoarylamino” and “diarylamino” containing one or two aryl radicals, respectively, attached to an amino radical. The term “aralkylamino”, embraces aralkyl radicals attached to an amino radical. The term aralkylamino denotes “monoaralkylamino” and “diaralkylamino” containing one or two aralkyl radicals, respectively, attached to an amino radical. The term aralkylamino further denotes “monoaralkyl monoalkylamino” containing one aralkyl radical and one alkyl radical attached to an amino radical.

As used herein, the term “heteroatom”, as used herein, refers to oxygen, sulfur, nitrogen, and phosphorus.

In some embodiments, the active compounds are present in the form of amines, and their pharmaceutically acceptable salts. Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, benzoate, and ascorbate; salts with amino acids such as lysine monohydrochloride, aspartate and glutamate. The salts may be in some cases hydrates or ethanol solvates. The salts can be prepared by reacting an active compound as described herein with a suitable acid. For transdermal administration, it can be preferred that the acid is a fatty acid, to form a salt that has relatively easy transmission through the skin.

In any embodiment described herein, the active blend of a dosage form generally includes one or more pharmaceutically acceptable adhesives, excipients, carriers, diluents, binders, lubricants, glidants, or disintegrants and depends upon the purpose for which the active ingredient is being applied. In general, transdermal formulations are made of other ingredients including, but not limited to, excipients, diluents, carriers, permeation enhancers, and mixtures thereof.

### Compounds

In some embodiments, the compounds described herein do not inhibit EPPIN-semenogelin binding, per se, but rather, bind to that portion of EPPIN to which anti-EPPIN antibodies bind. Representative anti-EPPIN antibodies are disclosed in O'Rand et al., Biology of Reproduction, 80, 279-285 (2009).

In other embodiments, the compounds described herein inhibit or enhance EPPIN-semenogelin binding, and inhibit forward motility and function of sperm in humans and other primates. The EPPIN-semenogelin complex is on the surface of sperm. Useful compounds include those that a) bind to the binding site on EPPIN for semenogelin (also referred to herein as SEMG), b) bind to an allosteric position in a manner which inhibits semenogelin from binding, and which also mimic the effect of the semenogelin, namely to stop sperm from swimming, and c) enhance the binding of semenogelin to EPPIN.

Ideally, those compounds which mimic the binding of the anti-EPPIN antibodies bind with higher affinity than the antibodies themselves, and/or those compounds which interfere with EPPIN semenogelin binding bind with higher affinity to the active binding pocket than semenogelin. This will enable one to administer lower effective concentrations of the compounds than compounds that bind with lower binding affinity. Those compounds which bind in an allosteric manner are also, ideally, high affinity compounds, so that lower effective concentrations of these compounds can be administered as well.

Compounds identified in the high-throughput assay as mimicking the binding of the anti-EPPIN antibodies, or inhibiting the binding of EPPIN and semenogelin, and also identified as having a negative impact on spermatozoa motility and function, can be used in the methods described herein.

The compounds of the present invention have the following structures and names:

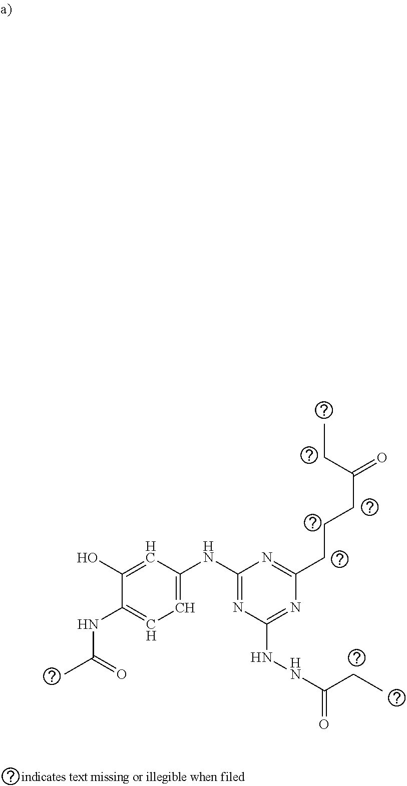 [SMILES: CCC(=O)CCCc1nc(NNC(=O)CC)nc(Nc2ccc(NC(C)=O)c(O)c2)n1]

### EP082: methyl 3-({4-[(4-acetamido-3-hydroxyphenyl)amino]-6-{[(methoxycarbonyl)amino]amino}-1,3,5-triazin-2-yl}sulfanyl)propanoate

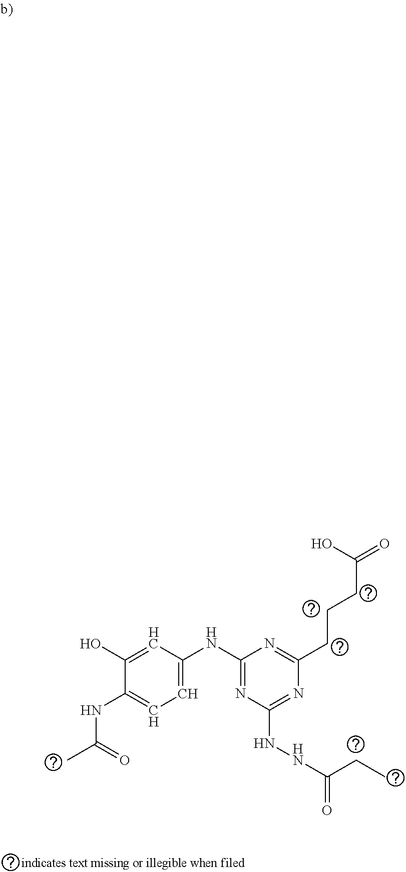 [SMILES: CCC(=O)NNc1nc(CCCC(=O)O)nc(Nc2ccc(NC(C)=O)c(O)c2)n1]

### EP083: 3-((4-((4-acetamido-3-hydroxyphenyl)amino)-6-(2-(methoxycarbonyl)hydrazineyl)-1,3,5-triazin-2-yl)thio)propanoic acid

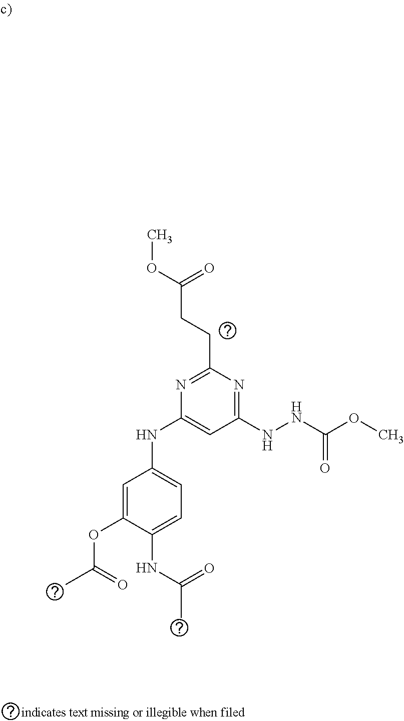 [SMILES: COC(=O)CCc1nc(NNC(=O)OC)cc(Nc2ccc(NC(C)=O)c(OC(C)=O)c2)n1]

### EP089: methyl 2-[(4{[3-(acetyloxy)-4 acetamidophenyl]amino}-6-{[(methoxycarbonyl)amino]amino}pyrimidin-2-yl)sulfa acetate

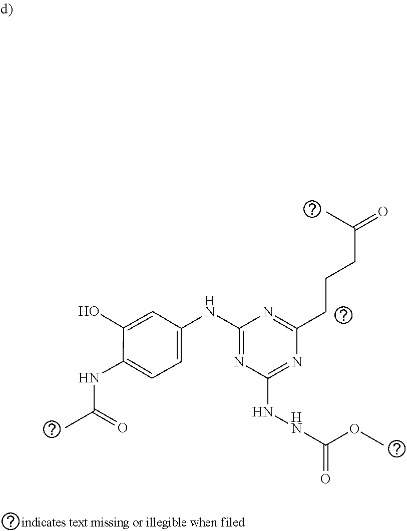 [SMILES: COC(=O)NNc1nc(CCCC(C)=O)nc(Nc2ccc(NC(C)=O)c(O)c2)n1]

### EP091: methyl 2-(4-((4-acetamido-3-hydroxyphenyl)amino)-6-((3-oxobutyl)thio)-1,3,5-triazin-2-yl) hydrazine-1-carboxylate

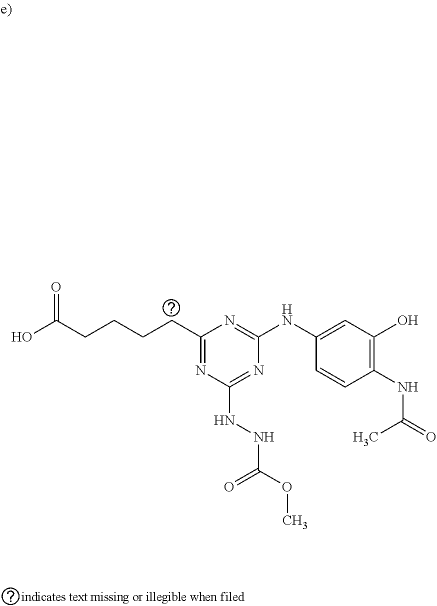 [SMILES: COC(=O)NNc1nc(CCCCC(=O)O)nc(Nc2ccc(NC(C)=O)c(O)c2)n1]

### EP096: 4-((4-((4-acetamido-3-hydroxyphenyl)amino)-6-(2(methoxycarbonyl)hydrazineyl)-1,3,5-triazin-2-yl)thio)butanoic acid

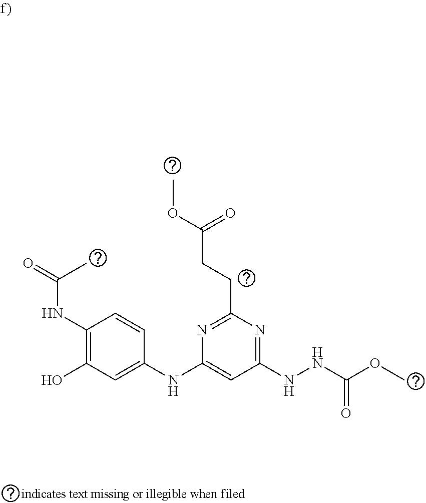 [SMILES: COC(=O)CCc1nc(NNC(=O)OC)cc(Nc2ccc(NC(C)=O)c(O)c2)n1]

### EP097: methyl 2-({4[(4 acetamido-3-hydroxyphenyl)amino]-6-{[(methoxycarbonyl)amino]amino}pyrimidin-2-yl}sulfan acetate

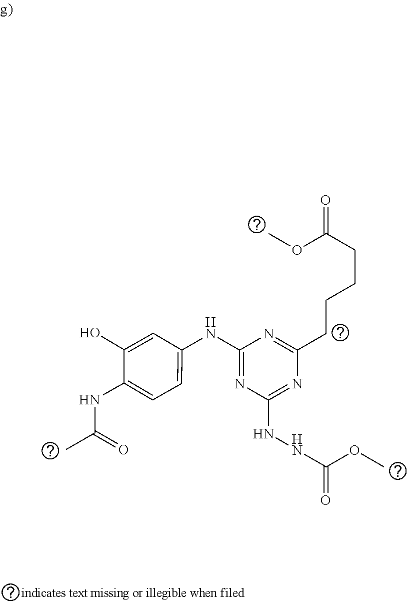 [SMILES: COC(=O)CCCCc1nc(NNC(=O)OC)nc(Nc2ccc(NC(C)=O)c(O)c2)n1]

### EP098: methyl 4-({4-[(4-acetamido-3-hydroxyphenyl)amino]-6-{[(methoxycarbonyl)amino]amino}-1,3,5-triazin-2-yl}sulfanyl)butanoate

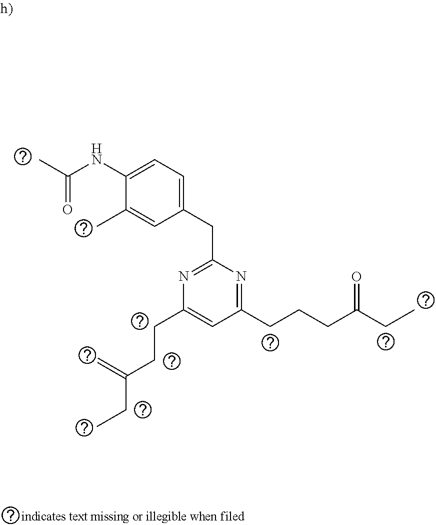 [SMILES: C=C(CC)CCc1cc(CCCC(=O)CC)nc(Cc2ccc(NC(C)=O)c(C)c2)n1]

### EP100: methyl 3-({2-[(4-acetamido-3-hydroxyphenyl)methyl]-6-{[(methoxycarbonyl)amino]amino}pyrimidin-4-yl}sulfanyl)propanoate

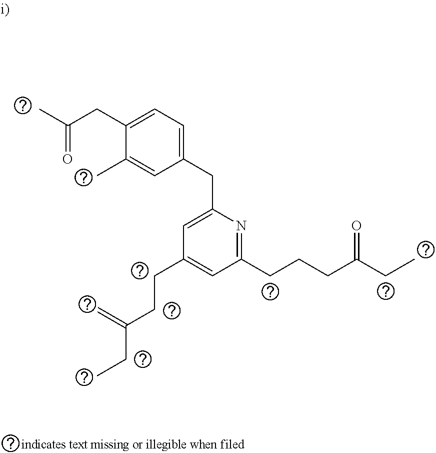 [SMILES: C=C(CC)CCc1cc(CCCC(=O)CC)nc(Cc2ccc(CC(C)=O)c(C)c2)c1]

### EP101: methyl 3-[(6-{[3-hydroxy-4-(2-oxopropyl)phenyl]methyl}-4-{[(methoxycarbonyl)amino]amino}pyridin-2-yl)sulfanyl]propanoate

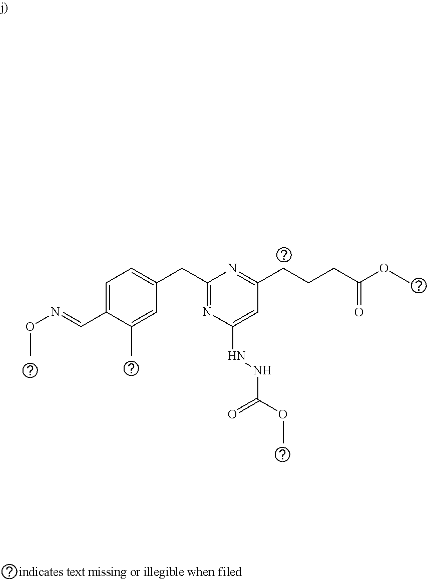 [SMILES: CO/N=C/c1ccc(Cc2nc(CCCC(=O)OC)cc(NNC(=O)OC)n2)cc1C]

### EP102: methyl 3-{[2-({3-hydroxy-4-[(1E)-(methoxyimino)methyl]phenyl}methyl)-6-{[(methoxycarbonyl)amino]amino}pyrimidin-4-yl]sulfanyl}propanoate

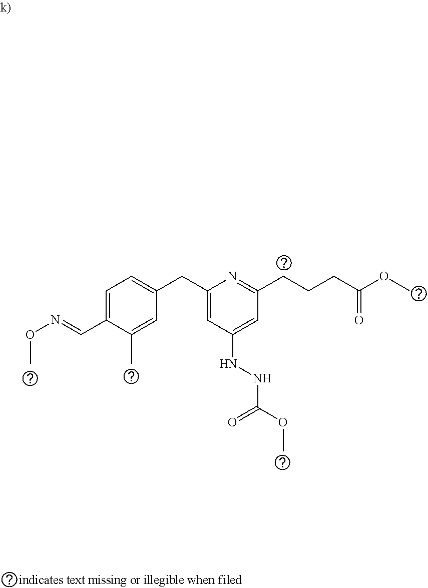 [SMILES: CO/N=C/c1ccc(Cc2cc(NNC(=O)OC)cc(CCCC(=O)OC)n2)cc1C]

### EP103: methyl 3-{[6-({3-hydroxy-4-[(1E)-(methoxyimino)methyl]phenyl}methyl)-4-{[(methoxycarbonyl)amino]amino}pyridin-2-yl]sulfanyl}propanoate

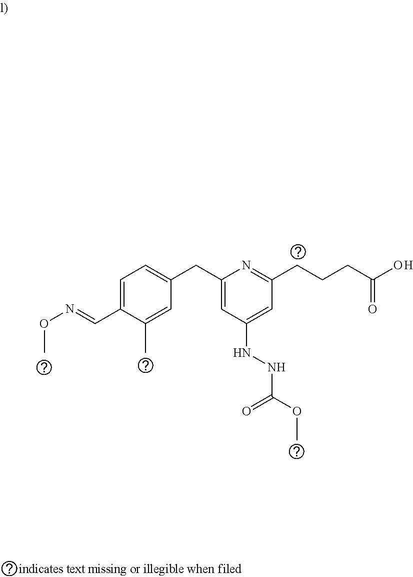

### EP104: 3-{[6-({3-hydroxy-4-[(1E)-(methoxyimino)methyl]phenyl}methyl)-4-{[(methoxycarbonyl)amino]amino}pyridin-2-yl]sulfanyl}propanoic acid

### Synthesis for Compounds EP082, EP083, EP091, EP096, and EP098

The compounds are prepared using the following general reactions:

a) Synthesis of Compound EP082

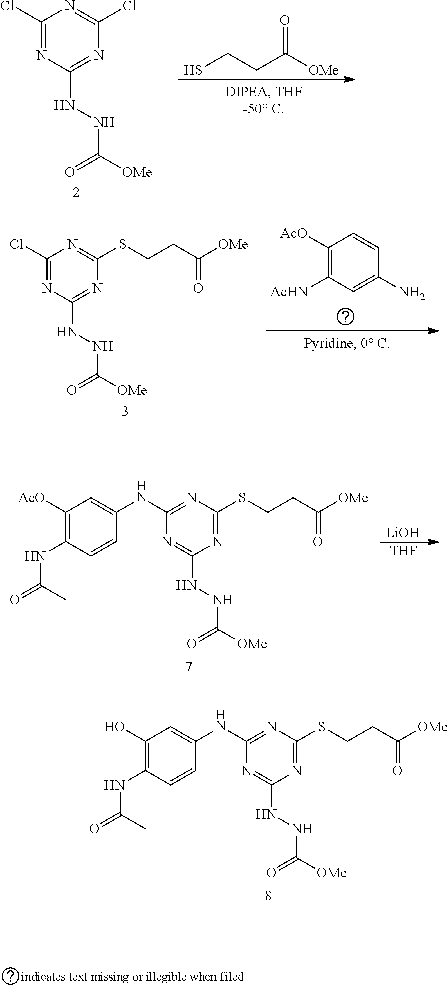

b) Synthesis of Compound EP083

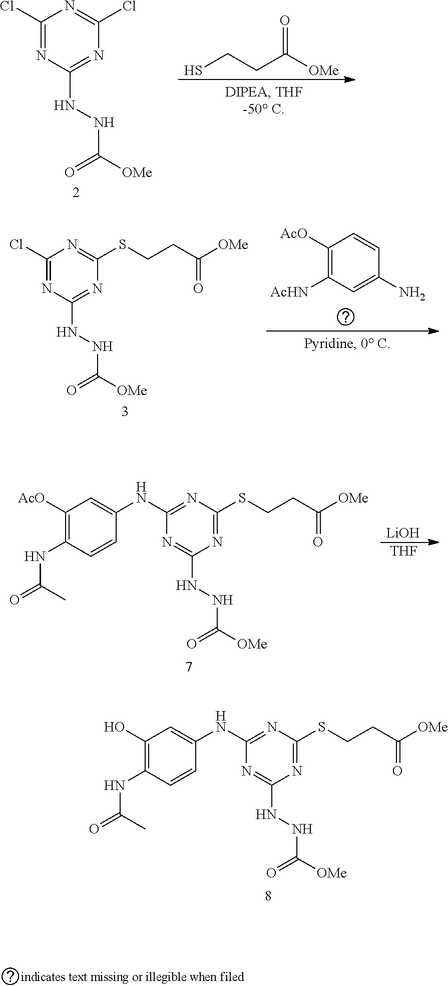

500 mg of compound 2→compound 3 after column purification obtained in 85% and 98% pure (575 mg). The reaction of 3 with 6 gave compound 7, which was purified on a biotage column to give >95% pure of 7 (drying under vacuum). Compound 7 was converted to compound 8 (EP083) with 6 eq. of LiOH/THF.

c) Synthesis of Compound EP091

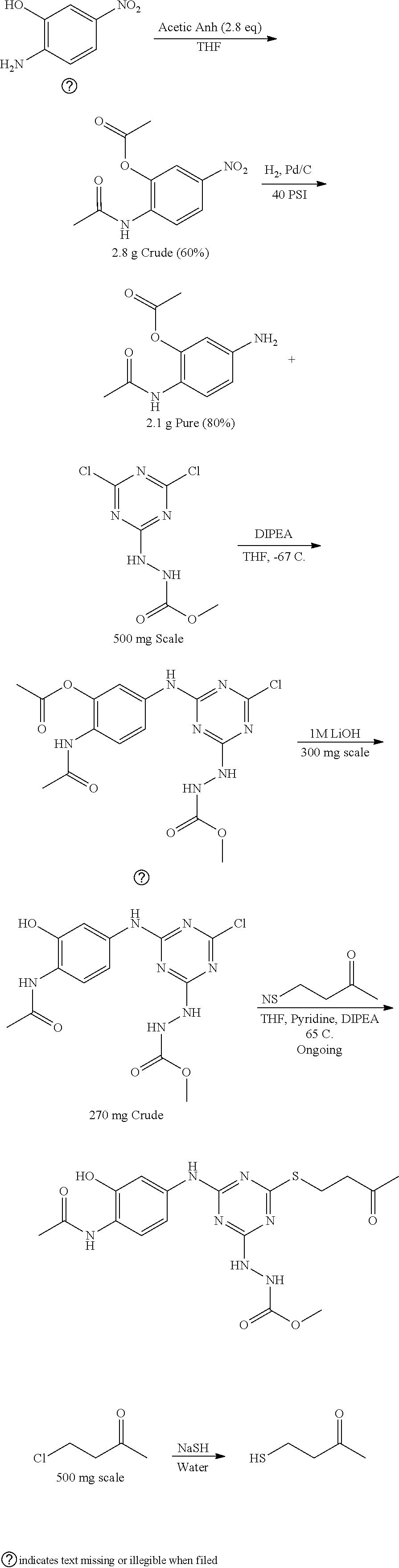

When 2 eq. of DIPEA was added and the temperature heated to 65 degree, product started to form. Heating was controlled on a timer for 18 h at 65 C. Reaction mixture gave 1:1 of 200 mg SM and product mixture. Prep. TLC: 12 mg of 90% pure product. Subsequent biotage: 2.5 mg of 99% purity.

d) Synthesis of Compound EP096

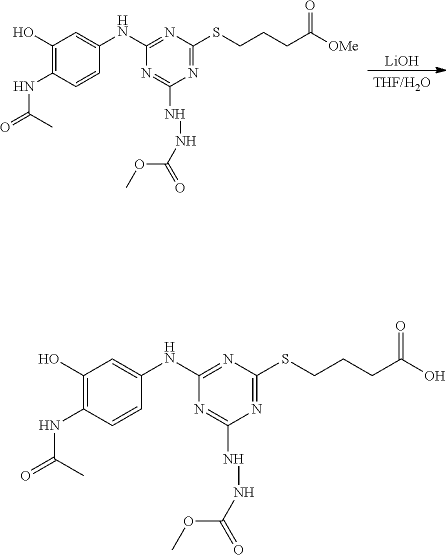

2 mg of 64 mg batch of ester was hydrolyzed to acid. The conversion was clean, carried out on 30 mg scale. After hydrolysis was done, the work up (acid/base extraction) gave >95% pure acid.

e) Synthesis of Compound EP098

Scaleup of thio-butyl ester below was carried out:

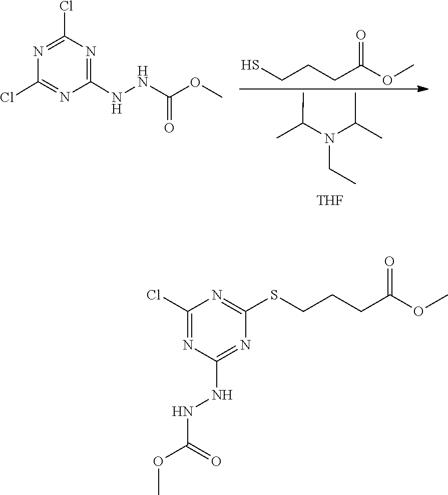

400 mg of pure thio butyl ester adduct was obtained.

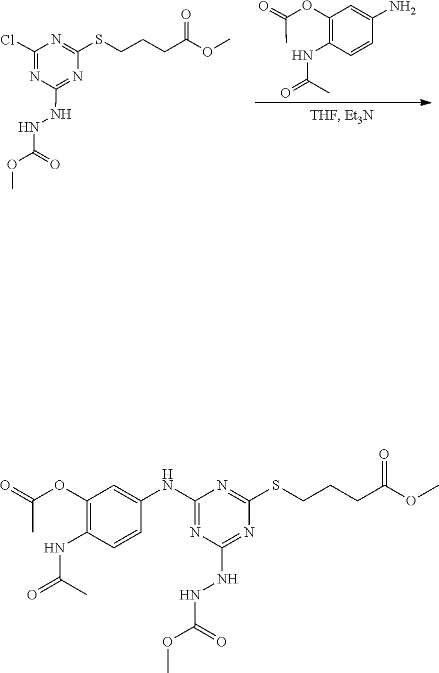 [SMILES: COC(=O)CCCSc1nc(Cl)nc(NNC(=O)OC)n1 ; CC(=O)Nc1ccc(N)cc1OC(C)=O ; COC(=O)CCCSc1nc(NNC(=O)OC)nc(Nc2ccc(NC(C)=O)c(OC(C)=O)c2)n1]

100 mg scale carried out overnight at 50° C. resulted in a mixture (SM was consumed, product+a number of by-products). Biotage purification gave 45 mg of 60% pure product, which was further purified on a prep TLC plate. The product band was separated and cut.

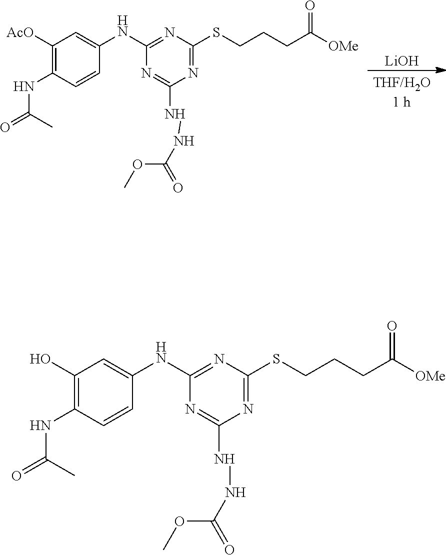

The 150 mg of 60% pure was deacylated with LiOH (2 eq.) in THF/H₂O at RT for 1 h shown above. The reaction mixture was purified on a biotage using 0-10% MeOH in DCM to give the desired final product with 8-10% less polar impurities, which (60 mg in total) was further purified on prep TLC.
pTLC purification was carried out twice. LCMS showed all impurities were removed. LCMS: 98%; 1H NMR: product peaks+residual hexane. 5.3 mg of the ester final product was dissolved in DCM/MeOH, and then precipitated from hexane. The solid was filtered and recovered to give 5 mg of product with 95% overall purity.

### Methods

Both the Epitope ALPHA assay and the live sperm CASA Assay were done by our standard procedures [1,2]

Briefly, recombinant histidine-tagged recombinant EPPIN was pre-incubated with Ni-NTA chelate donor beads (catalog number AS101, Perkin Elmer, Waltham MA) and anti-Eppin 007 or anti-Eppin Q20E antibody was pre-incubated with Protein A acceptor beads (catalog number 6760136, Perkin Elmer, Waltham MA) for 30 minutes. Equal volumes of donor and acceptor bead mixtures were pipetted into white opaque 384-well microplates (OptiPlate-384; PerkinElmer, Waltham, MA) in a final volume of 30 μL. Plates were covered with top seal and transferred to a Synergy HTS Multiplatform automated plate reader (Biotek, Winooski, VT). After shaking for 2 min, plates were read with an excitation 680/30 filter, an emission 570/100 filter and data acquired using Gen5 software (Biotek). Each set of samples was pipetted in 4 replicates. The final concentration of assay components was 58 nM EPPIN, 2 nM anti-Eppin 007 or Q20E antibody, 5 μg/ml donor beads and 10 μg/ml acceptor beads in 0.1M Tris pH=8, 0.1% bovine serum albumin and 0.01% Tween-20. Negative controls were performed under the same conditions in the absence of EPPIN or antibody and in the presence of beads only. The antibody 007 used in this assay binds the same epitope that binds the EP055 compound so the two entities compete for binding to EPPIN. Q20E binds the n-terminal part of EPPIN.

Non-specific interactions of compound with assay components were measured at the same time with the IgG detection kit (0.5 mM biotinylated rabbit IgG) to generate the bead interaction: 10 μg/ml donor and acceptor beads. (catalog number 6760617C, Perkin Elmer).

A specific signal for each time point was calculated by subtracting the background signal (obtained in the absence of Eppin) from its respective total signal. EC₅₀ values for the binding of EPPIN to specific compounds were calculated by non-linear regression curve fitting using the specific signal obtained after 16 h of incubation using GraphPad Prism version 6.00 for Windows (GraphPad Software, La Jolla California USA, www.graphpad.com).

The live-sperm Computer Assisted Sperm Analysis (CASA) Assays were done as follows.

Human semen samples were obtained from the Department of Obstetrics and Gynecology, University of North Carolina Memorial Hospital, Chapel Hill, NC. The Institutional Review Board determined that studies with these samples did not constitute human subjects research as defined under U.S. federal regulations [45 CFR 46.102 (d or f) and 21 CFR 56.102(c) (e) (I)] and the need for informed consent was waived. Semen samples were allowed to liquefy for 30 minutes and subjected to standard semen analysis to determine acceptability before freezing. All samples were stored in liquid nitrogen, and de-identified by the Department of Obstetrics and Gynecology, University of North Carolina Memorial Hospital, Chapel Hill, NC before further processing. A density gradient (SpermCare catalog number 2221 and 2222, InvitroCare Inc., Frederick MD) was used to prepare spermatozoa for further analysis. At least 3 independent experiments were done per assay, each using several pooled samples from a single donor.
Briefly sperm were washed after the density gradient with EP buffer-0.4% human serum albumin and resuspended in the same buffer for compound treatments. Sperm were diluted to 5×10⁶/mL in 12×75 mm glass tubes with the appropriate treatment and incubated at 37° C. in 5% CO₂. For CASA analysis, sperm were loaded onto slides (CellVison 4 chamber (20 μM), catalog number CV-1020-4CV, Fertility Technology Resources, Murphy NC) and warmed to 37° C. before analysis. Videos of sperm motility were collected using a Nikon ECLIPSE TS2-FL inverted microscope with 10× eyepiece and a 10× negative phase contrast objective and a Basler high speed camera (1280×1024 px, 200 fps, USB 3, CMOS). AVI Videos were analyzed using OpenCASA software (version 2) [3]. Units for CASA parameters are as follows: VAP (average path velocity), VSL (straight line velocity), and VCL (curvilinear velocity)=μm/sec (microns/sec); ALH (lateral head amplitude)=μm; BCF (beat cross-frequency)=Hz. The relative mobility index (RMI) is a measure of the effect of compound on sperm and is calculated as the VSL x % normal motility and normalized to the appropriate DMSO control.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials, and the like apparent to those skilled in the art still fall within the scope of the invention as claimed by the applicant.

What is claimed is:

1-4. (canceled)

5. A composition providing male contraception comprising:
4-((4-((4-acetamido-3-hydroxyphenyl)amino)-6-(2-(methoxycarbonyl)hydrazineyl)-1,3,5-triazin-2-yl)thio)butanoic acid.

6. The composition according to claim 5, wherein the composition is formulated into a capsule.
